(19) United States
(12) Patent Application Publication
(10) Pub. No.: US 2013/0267902 A1
(43) Pub. Date: Oct. 10, 2013

(54) Instrument Support Fixture

(71) Applicant: ARKIS, LLC, Knoxville, TN (US)

(72) Inventors: Chad Seaver, Knoxville, TN (US); James Alexander Killeffer, Knoxville, TN (US); Chris Arnott, Knoxville, TN (US)

(73) Assignee: Arkis, LLC, Knoxville, TN (US)

(21) Appl. No.: 13/858,614

(22) Filed: Apr. 8, 2013

Related U.S. Application Data
(60) Provisional application No. 61/621,511, filed on Apr. 7, 2012.

(51) Int. Cl. A61M 27/00

(52) U.S. Cl. CPC A61M 27/006
USPC 604/175

(57) ABSTRACT

An instrument support fixture, and a method of supporting an instrument using the fixture, the instrument support fixture including a first supporting portion configured to be adhered to an area of a patient, a coupling portion having first and second ends, the first end being coupled to the first supporting portion, and a second supporting portion coupled to the second end of the coupling portion to secure an instrument during a procedure.

### CROSS-REFERENCE TO RELATED APPLICATIONS

This application claims priority from U.S. Provisional Application No. 61/621,511, filed on Apr. 7, 2012.

### FIELD OF INVENTION

The present general inventive concept relates generally to systems and methods of supporting instruments during procedures such as treating hydrocephalus or the like, and, more particularly, to a surgical holding/positioning fixture to secure a ventricle catheter during a hydrocephalus shunt installation surgical procedure, and methods of carrying out the same.

### BACKGROUND

Conventionally, in the case of a hydrocephalus shunt surgical installation procedure with the shunt located at the top of the patient's skull, the placement of a ventricular catheter must be inserted into the patient's ventricles through an access hole drilled into the skull. Referring to FIG. 1, which is a simple schematic representation illustrating a conventional ventricle catheter installation, the ventricle catheter during a hydrocephalus shunt surgical installation procedure is generally indicated by 100. First the ventricle catheter entry site on the patent's skull 101 is prepared by shaving and sterilizing the patent's skin above the area where the skull bore hole entry will be made. Next an incision is made exposing the patent's skull, and then an access bore hole 102 is made through the patient's skull, exposing the patient's brain. Lastly the ventricular catheter 103, illustrated by a simple line in this illustration, is inserted through the bore hole 102 and is left substantially unsupported while the surgery progresses. Optionally, the surgeon may manually, in combination with a tool such as forceps, hold the catheter during the remaining procedure. An unsupported ventricle catheter poses significant risk to the success of the surgical procedure, such as the potential case of catheter loss into the patient's skull, and/or misplacement and/or misalignment. Also the use of a surgeon's hand to hold the catheter significantly reduces the surgeon's efficiency and prolongs the time required to complete the surgical procedure. Once the remaining surgical steps of placing the shunt valve, with optional control system, drainage tubing, and peritoneal catheter are complete, the tubing/valve is connected to the ventricle catheter. Afterwards, the ventricle catheter 103 is finally pushed into its final position and the surgery is completed. It will be appreciated by one skilled in the art that a method and/or apparatus is desired to secure the ventricle catheter during the shunt installation surgical procedure.

### BRIEF SUMMARY

The present general inventive concept provides an instrument support fixture, and a method of using same, to adhere to an area of the patient and support a medical instrument during a procedure such that a user's hands are free to perform other procedures.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by an instrument support fixture including a first supporting portion configured to be adhered to an area of a patient, a coupling portion having first and second ends, the first end being coupled to the first supporting portion, and a second supporting portion coupled to the second end of the coupling portion to secure an instrument during a procedure.

The coupling portion may be formed at least partially of a rigid or semi-rigid material so as to maintain a shape manually formed by a user.

The coupling portion may include a magnetic material to which one or more medical instruments may be adhered.

The first supporting portion may be semi-rigid or rigid and at least partially contoured to accommodate the curvature of the patient's head.

The first supporting portion may be formed of two or more contact members extending to different locations from the first end of the coupling portion to contact the area of the patient.

The first supporting portion may include one or more through holes to accommodate a screw to adhere the first supporting portion to the patient's head.

The screw may be a self-retaining or captive screw.

The instrument support fixture may further include a strap coupled to the first supporting portion to adhere the first supporting portion to the patient's head.

The strap may be formed of an elastic material.

The second supporting portion may be a clamp having a gripping portion to manually release and apply the clamp.

The instrument support fixture may further include a controller in communication with the second supporting portion to remotely release and apply a clamping action by the second supporting portion.

The controller may be configured as a foot operated switch that is coupled to the second supporting portion by a wired connection.

The second supporting portion may include a mechanical, electromechanical, and/or electromagnetic releasing mechanism to release and apply the clamping action according to the controller.

The first supporting portion may be a base portion configured to be adhered to the patient's head.

The second supporting portion may be a clamp coupled to the second end of the coupling portion to hold a catheter during a medical procedure.

The first supporting member may be configured to be inserted between the patient's scalp and skull.

The instrument support fixture may further include a securing pin configured to pass through the patient's scalp and couple to the first supporting member to anchor the instrument support fixture to the patient.

The first supporting portion may be configured in a curved shape so as to at least partially extend around at least a portion of a bore hole in which the instrument is to be inserted.

The first supporting portion may be provided with a plurality of through holes to receive a corresponding plurality of screws to adhere the first supporting portion to the patient.

At least a portion of the first supporting fixture may be bifurcated to form a receiving portion between top and bottom bifurcated portions to receive tissue of the patient to adhere the first supporting portion to the patient.

The foregoing and/or other aspects and advantages of the present general inventive concept may also be achieved by an instrument support fixture including a first supporting portion configured to be adhered to an area of a patient, and a second supporting portion having a first end coupled to the first supporting portion, and a second end extending away from the first supporting portion to secure an instrument during a procedure

The second supporting portion may extend horizontally from the first supporting portion such that the second end is adjacent to a bore hole in which the instrument is to be inserted.

The second end may be configured with a partially open and rigid or semi-rigid receiving portion such that the instrument is secured in response to being pushed into the receiving portion from a side adjacent to the receiving portion.

The receiving portion may be configured as a hook having a rigid or semi-rigid inner dimension to receive the instrument.

The foregoing and/or other aspects and advantages of the present general inventive concept may also be achieved by a method of supporting an instrument during a medical procedure, the method including adhering an instrument support fixture to a patient, the instrument support fixture having a first supporting portion configured to be adhered to an area of the patient, a second supporting portion configured to secure the instrument, and a coupling portion configured to couple the first supporting portion to the second supporting portion, and controlling the second supporting portion to selectively secure and release the instrument during the medical procedure.

The controlling of the second supporting portion may include hand-operating a gripping portion of the second supporting portion.

The controlling of the second supporting portion may include operating a gripping portion of the second supporting portion with a controller provided away from the instrument support fixture.

The controller may be a foot-switch operated by a user.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a conventional ventricle catheter installation;

FIG. 2 illustrates a catheter secured by an instrument supporting fixture according to an example embodiment of the present general inventive concept;

FIG. 3 illustrates the area of the conventional ventricle catheter installation of FIG. 1 in more detail;

FIG. 4 illustrates a more detailed example embodiment of the present general inventive concept;

FIG. 5 illustrates an instrument supporting fixture according to another example embodiment of the present general inventive concept;

FIG. 6 illustrates a controller for the instrument supporting fixture according to an example embodiment of the present general inventive concept;

FIG. 7 illustrates an instrument supporting fixture according to yet another example embodiment of the present general inventive concept; and

FIG. 8 illustrates an instrument supporting fixture according to still another example embodiment of the present general inventive concept.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Various example embodiments of the present general inventive concept, as described herein, provide an instrument support fixture, and a method of using the fixture, the fixture including a first supporting portion configured to be adhered or attached to an area of a patient, a coupling portion having first and second ends, the first end being coupled to the first supporting portion, and a second supporting portion coupled to the second end of the coupling portion to secure an instrument during a procedure. Various terms may be used interchangeably throughout these descriptions to refer to some elements. For example, the first supporting portion may be referred to as a base, the coupling portion may be referred to as a connecting portion, and the second supporting portion may include or be referred to as a holding fixture, clamp, etc., in various portions of the descriptions of these example embodiments. Various example embodiments of the present general inventive concept overcome the previously described limitations of the conventional hydrocephalus shunt installation surgical procedure, as well as other limitations, with a ventricle catheter holding fixture to secure the ventricle catheter and increase surgical productivity by allowing the surgeon to focus on the remaining surgical steps.

Although various example embodiments of the present general inventive concept generally describe an instrument support fixture to support a wide variety of medical instruments, the descriptions herein generally describe a method and device to secure a ventricular catheter during a hydrocephalus shunt surgical procedure.

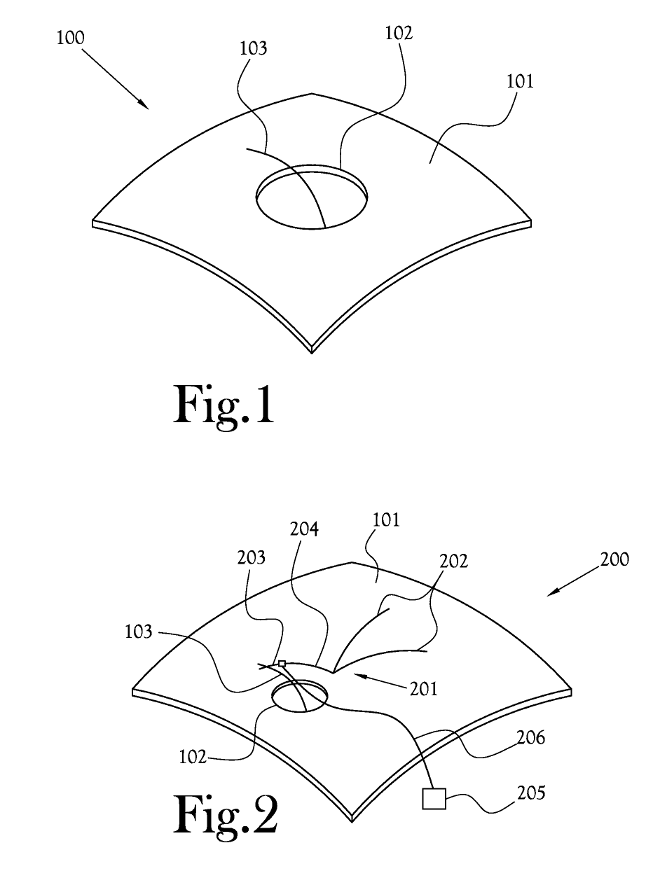

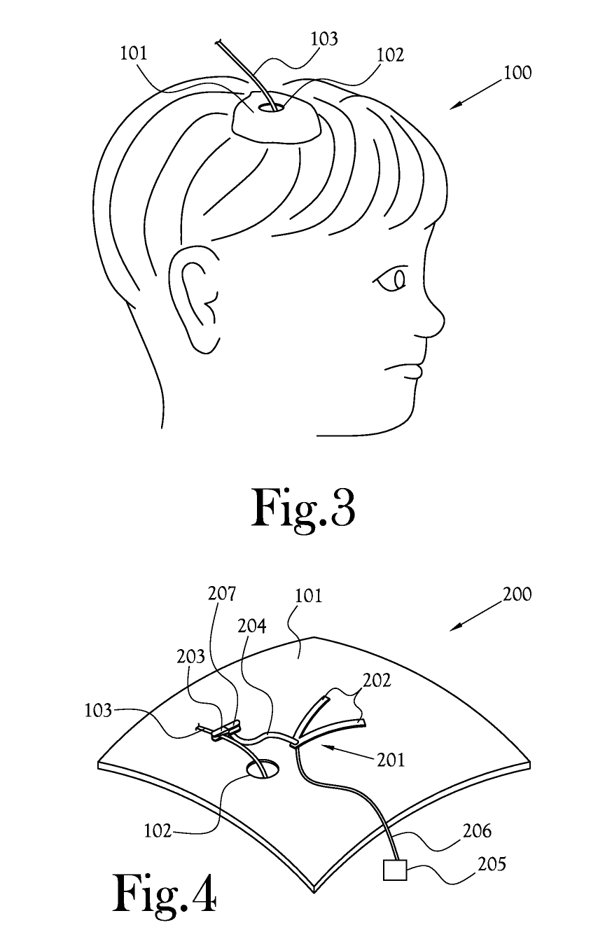

FIG. 2 illustrates an instrument, in this example a ventricle catheter, secured by an instrument supporting fixture according to an example embodiment of the present general inventive concept. Various other example embodiments of the present general inventive concept can be used to securely hold medical instruments other than a ventricle catheter, and during procedures other than a hydrocephalus shunt installation procedure, in which the secure holding of a device during a medical procedure would be beneficial. FIG. 3 illustrates the area of the conventional ventricle catheter installation of FIG. 1 in more detail, so as to provide more perspective of the area 101 illustrated in FIGS. 1 and 2. In several of the figures, the illustration of the area of the patient's skull 101 is presented without the patient's skin for the purposed of simplifying the drawing. It is understood that in a typical procedure an incision has been made above the subject area of the patient's skull and fixed so as to remain open to allow access to the area 101.

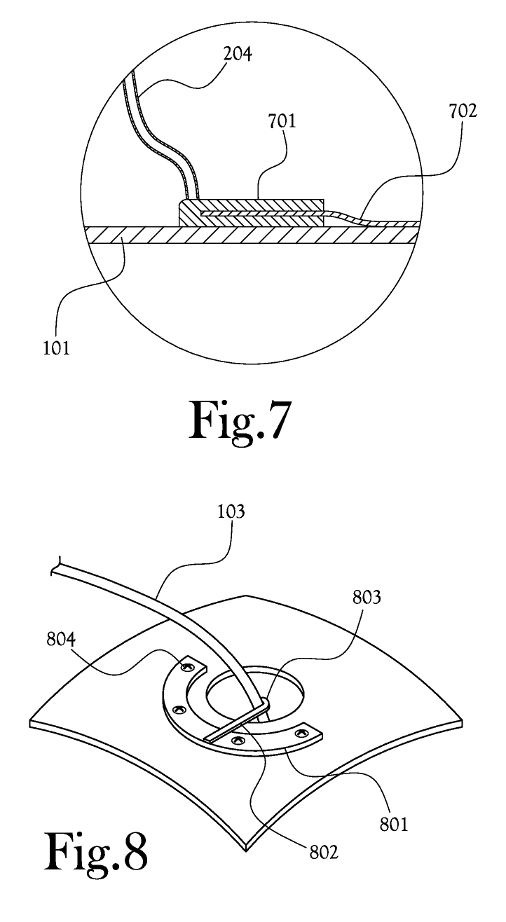

Referring to FIG. 2, a schematic representation of a ventricle catheter installation with an example embodiment of a holding fixture 201 during a hydrocephalus shunt surgical installation procedure is generally indicated by 200. First the ventricle catheter entry site and bore hole 102 is prepared, then the ventricle catheter 103 is inserted through the bore hole 102 and is left substantially supported by an instrument supporting fixture 201 having a base 202 attached to a ventricle catheter clamp 203 though a semi-ridged connection 204. The connection 204, which couples the base 202 to the clamp 203, may be rigid or semi-rigid, according to various example embodiments of the present general inventive concept, so as to maintain a shape/configuration formed by a user that bends the connection 204 to a desired position. The base 202 is adhered to the patient during the medical procedure. The base 202 may be securely attached/anchored to the patient with staples, adhesive, screws, wrap-around clamp, a strap, etc., such that the base 202 may be located away from the bore hole 102 for substantially unrestricted access during the procedure. In other various example embodiments, the base may be at least partially adhered to the patient by inserting a portion of the base between the scalp and the skull of the patient, one example embodiment of which will be described later in FIG. 7. In various example embodiments such as the example illustrated in FIG. 7, the portion located under the scalp may have a corresponding portion fitting over the scalp in, for example, a bobby-pin fashion. In other various example embodiments in which a portion of the base is inserted between the scalp and the skull of the patient, the base may be further secured by placing a needle through the scalp and at least a portion of the base to anchor the instrument supporting fixture during the procedure. The needle may also interact with a needle receiving portion of the base 202.

According to various example embodiments of the present general inventive concept, the base 202 may be at least partially contoured to accommodate the curvature of the patient's head, and/or may be formed of two or more contact members extending in different directions, or to different locations, from the first end of the connection 204, at which the connection 204 is coupled to the base 202, to contact an area of the patient. In various example embodiments, the base 202 may include one or more through holes to accommodate one or more screws, such as, for example, self-retaining captivated screws, to adhere the base to the patient's head or other body area. The fixture 201 may further include a strap, which may be formed of an elastic material, coupled to the base to adhere the base to the patient's head.

FIG. 4 illustrates a more detailed example embodiment of the present general inventive concept. For example, FIG. 4 illustrates the clamp 203 as having manual grips 207 that may be operated by a user to apply and release the clamp's pressure on the catheter 103. In other various example embodiments, the surgeon may manually operate the clamp 203 through a controller such as an optional remotely controlled device 205 such as a mechanical/electrical foot pedal in electrical communication with the holding fixture 201 by a wired connection 206, or other such remote controlled device in wired or wireless communication with the holding fixture 201 to open and close or otherwise manipulate the clamp 203.

The clamp 203 or the connection 204 can also be configured to include a magnetic material such that a magnetic attachment of the catheter or other instruments is possible. FIG. 5 illustrates a instrument supporting fixture according to another example embodiment of the present general inventive concept. In the example embodiment illustrated in FIG. 5, a magnetic material 501 is included in the connection 204, either as an integral part of the connection 204 or an separate component provided to the connection 204, and a variety of medical instruments 502,503 are magnetically adhered to the magnetic material for easy access by the user during the medical procedure.

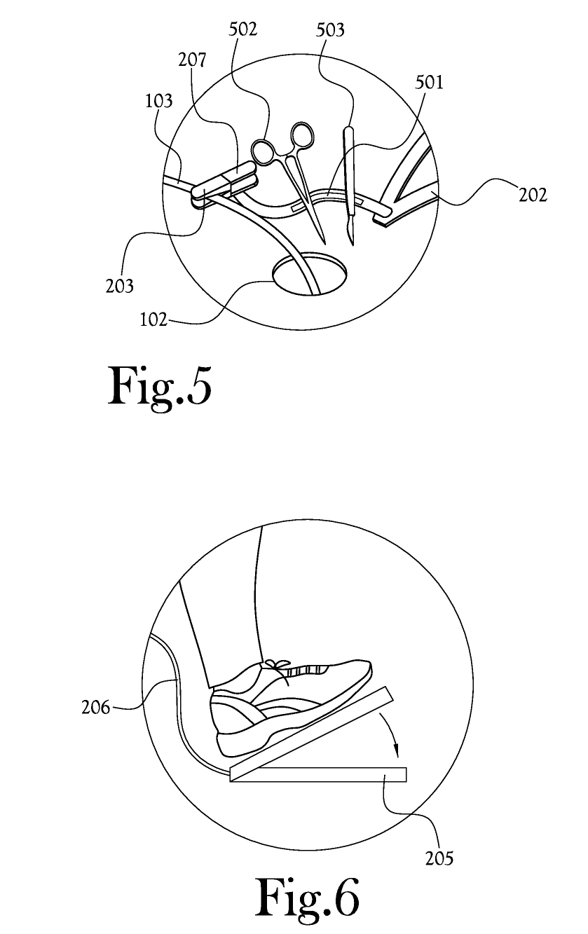

FIG. 6 illustrates a controller for the instrument supporting fixture according to an example embodiment of the present general inventive concept. In this example embodiment illustrated in FIG. 6, the controller 205 is configured as a foot pedal which may be operated by the user's foot, and is connected to the holding fixture 201 by the wired connection 206. A variety of other remotely located controllers may be provided, which may be operated by hand, foot, or other such actions. Also, it is understood that a foot-operated switch may be in any of a variety of configurations, and is not limited to the foot pedal illustrate in FIG. 6. The control by the controller 205 may also be applied to the holding fixture 201 by a wireless connection, and the releasing and application of the grip of the clamp 203 may be by an electromechanical element, an electromagnetic element, and so on.

FIG. 7 illustrates a cross section of an instrument supporting fixture according to yet another example embodiment of the present general inventive concept. In the example instrument support fixture illustrated in FIG. 7, the first supporting portion or base 701 having at least a portion that is bifurcated to form a receiving portion between the top and bottom bifurcated portions. By sliding this base 701 toward the edge of the skin 702 of the patient in which the incision has been made such that the skin 702 is received into the receiving portion of the base 701, the skin's natural adherence to the patient's skull provides an adhering force to adhere the base 701 to the patient. Such a configuration allows the base to be moved further away from the entry point of the instrument into the patient, and/or allows the area which must be exposed by the incision to be smaller since the base 701 will be partially placed above and below the skin. The base 701 may be additionally adhered to the patient by the previously described screws, adhesive, staples, etc. Also, as previously described, other example embodiments of the present general inventive concept may provide a base with a portion that slips under the skin but does not have the corresponding portion above the skin. Some such examples may be further adhered to the patient by a fixing member such as a needle that is passed through the skin to the base.

FIG. 8 illustrates an instrument supporting fixture according to yet another example embodiment of the present general inventive concept. In the example embodiment of the instrument support fixture illustrated in FIG. 8, the first supporting fixture or base 801 is configured in a curved shape, such as, for example, a “U” or horseshoe shape, so as to at least partially extend around at least a portion of the bore hole 102 in which the instrument or catheter 103 is inserted. The base 801 may be provided with a plurality of through holes 804 to receive a corresponding plurality of screws to adhere the base 801 to the patient. In various example embodiments, the base 801 may be previously provided with screws that are “captured,” or configured to maintain a presence in the through holes so as not to fall out during manipulation of the base or instrument supporting fixture. In the example embodiment illustrated in FIG. 8, a second supporting portion 802 has a first end coupled to the first supporting portion by any of several possible coupling methods/materials, and a second end extending away from the first supporting portion to secure the catheter 103 or other instrument during a procedure. Therefore, rather than having a rigid or semi-rigid coupling portion between the first and second supporting portions, as seen in various other example embodiments described herein, in the example embodiment illustrated in FIG. 8 the second supporting portion 802 is coupled directly to the first supporting portion 801. The second supporting portion 802 may be configured as a rigid or semi-rigid member, and may have a variety different securing configurations with which to secure an instrument.

In the example embodiment illustrated in FIG. 8, the second supporting portion 802 extends horizontally from the first supporting portion 801 such that the second end is adjacent to the bore hole in which the catheter 103 or other instrument is to be inserted. However, other various example embodiments are not limited to such a configuration. In the example illustrated in FIG. 8, the second end is provided with a securing configuration having a partially open and flexible, or semi-rigid, receiving portion such that the catheter 103 is secured in response to being pushed into the receiving portion from a side adjacent to the receiving portion. In other words, a user may “snap” the catheter 103 or other instrument into the receiving portion, and the catheter 103 will remain secured until the user removes the catheter 103 from receiving portion of the second supporting portion. In various example embodiments the receiving portion may be configured as a hook having an inner diameter that expands to receive the catheter 103 or other instrument. In other words, in various example embodiments, the flexible nature of the receiving portion allows the hook to expand to accommodate the larger catheter 103 and hold it securely, and then allows the user to conveniently remove the catheter 103 by pushing it out of the hook portion. In some example embodiments, the receiving portion may be configured to be rigid or semi-rigid such that the catheter 103 or other instrument contracts to the inner dimension of the receiving portion in response to the user pushing the catheter 103 into the receiving portion.

Various example embodiments of the present general inventive concept provide a method of supporting an instrument, such as a catheter, during a procedure, the method including adhering the instrument support fixture to the patient, the instrument support fixture having a first supporting portion, such as a base, configured to be adhered to an area of the patient, such as a portion of the skull. The securing and releasing of the instrument is controlled during the medical procedure by manipulating a second supporting portion that is configured to secure the instrument, the position of the second supporting portion being adjustable by the user by a rigid or semi-rigid coupling portion which couples the first supporting portion to the second supporting portion.

Thus the holding fixture 201 securely holds the ventricle catheter during the surgical procedure, which prevents catheter loss and possible misalignment. It is also recognized that other devices and/or attachments can be connected to the fixture such as lights, tools, or other devices that can aid during the surgical procedure. Furthermore, since the holding fixture offers a secure and stable platform it can also be used to support other instruments and/or measurements and/or even be used as a reference point for geospatial alignment.

According to various embodiments of the present general inventive concept, an instrument support fixture, and a method of using same, to adhere to an area of the patient and support a medical instrument during a procedure such that a user's hands are free to perform other procedures, is provided.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. An instrument support fixture comprising:
a first supporting portion configured to be adhered to an area of a patient;
a coupling portion having first and second ends, the first end being coupled to the first supporting portion; and
a second supporting portion coupled to the second end of the coupling portion to secure an instrument during a procedure.

2. The instrument support fixture of claim 1, wherein the coupling portion is formed at least partially of a rigid or semi-rigid material so as to maintain a shape manually formed by a user.

3. The instrument support fixture of claim 1, wherein the coupling portion comprises a magnetic material to which one or more medical instruments may be adhered.

4. The instrument support fixture of claim 1, wherein the first supporting portion is semi-rigid or rigid and at least partially contoured to accommodate the curvature of the patient's head.

5. The instrument support fixture of claim 1, wherein the first supporting portion is formed of two or more contact members extending to different locations from the first end of the coupling portion to contact the area of the patient.

6. The instrument support fixture of claim 1, wherein the first supporting portion includes one or more through holes to accommodate a screw to adhere the first supporting portion to the patient's head.

7. The instrument support fixture of claim 6, wherein the screw is a self-retaining or captive screw.

8. The instrument support fixture of claim 1, further comprising a strap coupled to the first supporting portion to adhere the first supporting portion to the patient's head.

9. The instrument support fixture of claim 8, wherein the strap is formed of an elastic material.

10. The instrument support fixture of claim 1, wherein the second supporting portion is a clamp having a gripping portion to manually release and apply the clamp.

11. The instrument support fixture of claim 1, further comprising a controller in communication with the second supporting portion to remotely release and apply a clamping action by the second supporting portion.

12. The instrument support fixture of claim 11, wherein the controller is configured as a foot operated switch that is coupled to the second supporting portion by a wired connection.

13. The instrument support fixture of claim 11, wherein the second supporting portion includes a mechanical, electromechanical, and/or electromagnetic releasing mechanism to release and apply the clamping action according to the controller.

14. The instrument support fixture of claim 1, wherein the first supporting portion is a base portion configured to be adhered to the patient's head.

15. The instrument support fixture of claim 1, wherein the second supporting portion is a clamp coupled to the second end of the coupling portion to hold a catheter during a medical procedure.

16. The instrument support fixture of claim 1, wherein the first supporting member is configured to be inserted between the patient's scalp and skull.

17. The instrument support fixture of claim 16, further comprising a securing pin configured to pass through the patient's scalp and couple to the first supporting member to anchor the instrument support fixture to the patient.

18. The instrument support fixture of claim 1, wherein the first supporting portion is configured in a curved shape so as to at least partially extend around at least a portion of a bore hole in which the instrument is to be inserted.

19. The instrument support fixture of claim 18, wherein the first supporting portion is provided with a plurality of through holes to receive a corresponding plurality of screws to adhere the first supporting portion to the patient.

20. The instrument support fixture of claim 1, wherein at least a portion of the first supporting fixture is bifurcated to form a receiving portion between top and bottom bifurcated portions to receive tissue of the patient to adhere the first supporting portion to the patient.

21. An instrument support fixture comprising:
a first supporting portion configured to be adhered to an area of a patient; and
a second supporting portion having a first end coupled to the first supporting portion, and a second end extending away from the first supporting portion to secure an instrument during a procedure.

22. The instrument support fixture of claim 21, wherein the second supporting portion extends horizontally from the first supporting portion such that the second end is adjacent to a bore hole in which the instrument is to be inserted.

23. The instrument support fixture of claim 21, wherein the second end is configured with a partially open and rigid or semi-rigid receiving portion such that the instrument is secured in response to being pushed into the receiving portion from a side adjacent to the receiving portion.

24. The instrument support fixture of claim 23, wherein the receiving portion is configured as a hook having a rigid or semi-rigid inner dimension to receive the instrument.

25. A method of supporting an instrument during a medical procedure, the method comprising:
adhering an instrument support fixture to a patient, the instrument support fixture having a first supporting portion configured to be adhered to an area of the patient, a second supporting portion configured to secure the instrument, and a coupling portion configured to couple the first supporting portion to the second supporting portion; and
controlling the second supporting portion to selectively secure and release the instrument during the medical procedure.

26. The method of claim 25, wherein the controlling of the second supporting portion comprises hand-operating a gripping portion of the second supporting portion.

27. The method of claim 25, wherein the controlling of the second supporting portion comprises operating a gripping portion of the second supporting portion with a controller provided away from the instrument support fixture.

28. The method of claim 27, wherein the controller is a foot-switch operated by a user.
